(19) United States
(12) Patent Application Publication
(10) Pub. No.: US 2006/0079143 A1
(43) Pub. Date: Apr. 13, 2006

(54) Controlled dosing of fibrous materials

(75) Inventors: Tuan T. Phan, Cary, IL (US); Gary L. Schweitzer, McHenry, IL (US)

Correspondence Address: BANNER & WITCOFF, LTD., TEN SOUTH WACKER DRIVE, SUITE 3000, CHICAGO, IL, 60606, US

(73) Assignee: Sage Products, Inc., Cary, IL (US)

(21) Appl. No.: 11/251,993

(22) Filed: Oct. 17, 2005

Related U.S. Application Data
(63) Continuation-in-part of application No. 10/435,898, filed on May 12, 2003.
Application No. 10/435,898 is a continuation-in-part of application No. 10/435,902, filed on May 12, 2003.

(51) Int. Cl. B05D 3/00; B32B 5/02

(52) U.S. Cl. 442/59; 427/2.31; 427/289

(57) ABSTRACT

Systems and methods relating to a properly dosed fabric, such as non woven fiber, are provided. In one aspect, the invention relates to a method for manufacturing a cloth having a consistent amount of medication thereon. In one embodiment, the weight and dimensions of the raw materials and the resulting cloths are compared to adjust the amount of solution that is applied to the fabric. In another embodiment, a continuous sheet of fabric is divided at select locations depending on its weight and physical dimensions. In one such embodiment, a controller may be utilized to determine the amount of tension to apply on the continuous non-woven fabric. In yet another embodiment, the tension applied to the fabric alters the physical dimensions of the fabric. Another aspect of the invention relates to a cloth of fibrous material, such as a non-woven cloth having a predetermined quantity of medication disposed thereon. In one embodiment, the cloth may be produced from a substantially continuous fabric

### PRIORITY INFORMATION

The present application is a continuation-in-part and claims priority to U.S. non-provisional application Ser. No. 10/435,898, filed May 5, 2003, and U.S. non-provisional application Ser. No. 10/435,902, filed May 5, 2003.

### FIELD OF THE INVENTION

The present invention relates to fabrics and cloths of fibrous material. More particularly, the invention provides systems and methods relating to consistent dosing of fiber, such as non-woven fabric, with a liquid solution.

### DESCRIPTION OF THE RELATED ART

Textiles have long been used as a medium for delivering liquid solutions. Increasingly, the medical, pharmaceutical, and related industries have utilized highly absorbent natural and synthetic fabrics to deliver medications and treatments. Under most current manufacturing processes, the blended cloths or fibers are supersaturated with the liquid preparation comprising the medication or substance to be delivered to the patient. The excess may then be removed by applying pressure to the cloth, such as through a squeezing mechanism. Unfortunately, the variance in the amount of medication throughout different regions of the fibers does not allow the accurate delivery of many preparations through this method. This problem is further exacerbated due to variable weight and dimensions of the cloths.

The weight of fabrics and especially non-woven fabrics, can be difficult to determine. Due to the manufacturing process for non-woven fabrics, many non-woven fabrics contain non-uniformities within the fabric. Different amounts of fibers may be present in different sections of the fabric. In addition, the process of forming non-woven fabrics can produce fabric with different basis weights and thicknesses from the middle than to the sides. Where non-woven fabrics are placed into a roll or other continuous lot of fabric, it can be difficult to weigh each separate inch of the fabric without cutting the fabric.

As more medications are administered through fabrics or fibrous materials, it is becoming increasingly important to determine the amount of medication or pharmaceutical preparation that is actually on the textile being used. For example, if the pharmaceutical preparation that may be used to disinfect at least a portion of a body is not properly dosed, the patient may not receive the desired benefit from the product. Indeed, if the cloth is under dosed, the patient may be placed at an elevated risk for developing an infection. Moreover, too much medication on the cloth may result in uneven exposure, unhealthy side effects, and increased cost of producing the product. Therefore, there exists a need in the art for methods and systems to accurately dose a fabric that will not substantially diminish its speed of manufacturing.

### SUMMARY OF THE INVENTION

The present invention overcomes at least some of the problems and limitations of the prior art by providing systems and methods that allow for a properly dosed fabrics or other substrates. In one aspect, the invention relates to a method for manufacturing a substrate such as a cloth, such as a non-woven cloth, having a pre-determined consistent dose of solution thereon. In one embodiment, the weight and dimensions of the raw materials and the resulting cloths are compared to adjust the amount of solution that is applied to the fabric. In another embodiment, a continuous sheet of fabric is divided at select locations depending on its weight and physical dimensions. In one such embodiment, a controller may be utilized to determine the amount of tension to apply on the continuous non-woven fabric. In yet another embodiment, the tension applied to the fabric alters the physical dimensions of the fabric.

Another aspect of the invention relates to a cloth of fibrous material, such as a non-woven cloth having a predetermined quantity of chemical compound such as a medication or pharmaceutical preparation disposed thereon. In one embodiment, the cloth may be produced from a substantially continuous fabric. In another embodiment, the cloth has an equal length-to-width ratio. Other features and advantages of the invention will be apparent from the description and drawings, and from the claims.

Of course, the methods and systems disclosed herein may also include other additional elements, steps, components, or raw materials.

The details of these and other embodiments of the present invention are set forth in the accompanying drawings and the description below.

### BRIEF DESCRIPTION OF THE DRAWINGS

The present invention may take physical form in certain parts and steps, a few embodiments of which will be described in detail in the following description and illustrated in the accompanying drawings that form a part hereof, wherein:

FIG. 1 shows a flowchart of one method of manufacturing a non-woven cloth having a solution according to one embodiment of the invention;

FIG. 2 is a diagram showing an illustrative system for manufacturing a non-woven cloth having a solution according to at least one embodiment of the invention; and

FIGS. 3a and 3b show the division of the substantially continuous fabric according to at least one embodiment of the invention.

Before the embodiments of the invention are explained in detail, it is to be understood that the invention is not limited in its application to the details of construction and the arrangement of the components set forth in the following description or illustrated in the drawings. The invention is capable of other embodiments and of being practiced or being carried out in various ways. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof.

### DETAILED DESCRIPTION

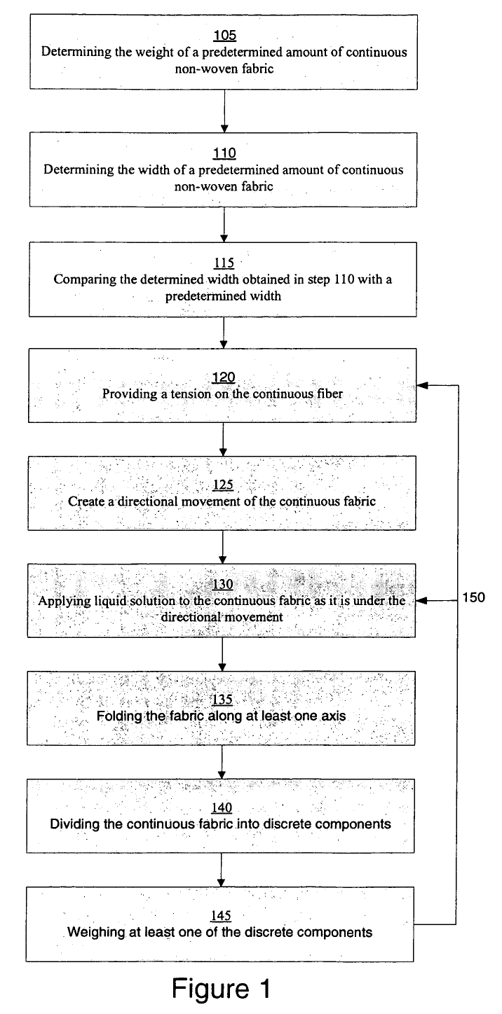

FIG. 1 shows a flowchart of one method of manufacturing a non-woven cloth having a predetermined consistent dose of solution thereon according to one embodiment of the invention. As used herein, the term “consistent” means within a predetermined range such as plus or minus 20% from a target value. As seen in the figure, the process may initiate with step 105 which determines the weight of a predetermined amount of continuous woven fabric. As used herein, the terms “continuous” and “substantially continuous” do not mean infinitely continuous. These terms mean materials or processes with substantial amounts or lengths. For example, a “continuous” or “substantially continuous” non-woven fabric connotes a fabric that has a length that is substantially greater than its width. In one embodiment a “continuous” fabric may be a roll of more than 50 feet of fabric with a width of less than 1 foot. As a further example, the term “continuous process” means a process that carries on for a period of time but is not infinitely continuous and is subject to stopping or pausing. The non-woven fabric is configured to be divided into smaller portions or cloths for one or more intended uses. The cloths may simply be portions of the fabric that have been divided or cut from the fabric. The continuous fabric may be in the form of a long overlapping sheet, a roll, or any configuration that allows for it to be readily utilized in a manufacturing process, but is not literally continuous.

Substrates other than non-woven cloths may be used in the invention. Substrates such as other fabrics, such as woven fabrics, may be used in the invention. In addition, substrates such as foam or sponge can be used in the invention. These substrates, in some embodiments of the invention, can be produced in the substantially continuous manufacturing processes described herein.

A non-woven fabric substrate, in an embodiment, is comprised of synthetic fibers, natural fibers or a combination of synthetic and natural fibers. Synthetic and natural fibers can include polyester, lyocell, polypropylene, rayon, cotton, acrylic, nylon, any plasticized cellulose acetate (PCA), additional fibers and blends of more than one of these fibers. These synthetic fibers can vary in length and thickness. In one embodiment, fibers can be used with lengths of approximately 0.5-5 inches, preferably approximately 1.5-3.0 inches. Fibers can be used with thicknesses of approximately 0.5-6 denier, preferably approximately 1.2-4.75 denier in an embodiment. The blend may be comprised of two or more multiple fibers in varying mixture ratios. In one embodiment, the non-woven fabric can be comprised of a blend of two polyester fibers. Approximately 60-80% of the polyester fibers have a denier of approximately 1.0-1.4, and more preferably approximately 1.2, and a length of approximately 1.2-1.8 inches and more preferably approximately 1.5 inches. Approximately 20-40% of the polyester fibers have a denier of approximately 4.5-5.0, preferably approximately 4.75, and a length of approximately 2.7-3.3 inches, preferably 3.0 inches. The tensile strength of the cloth in an embodiment is greater than approximately 27 lb./in.

In one embodiment, the continuous fabric is a roll substantially comprised of polyester fibers, and commercially available from any number of nonwoven manufacturers. Yet in other embodiments, a blend of different materials may be utilized. As one skilled in the art will readily appreciate, any number of fabrics may be used in one or more of the embodiments of the invention, in which the uses of specific fibers may be dictated by the intended use of the final product.

Determining the weight of the continuous fabric may be performed through one or more of a plurality of mechanisms. In one embodiment, the determination of the weight of the continuous fabric is accomplished by predicting the weight of the fabric. In an embodiment, the average known weight of previously utilized amount of fabric may be used. For example, if a manufacturing facility routinely receives a roll of continuous fabric that consistently has substantially the same weight, the manufacturer may utilize that value with known variances as the weight. In another embodiment, the actual continuous fabric to be used in the manufacturing process may be weighed. In yet another embodiment, only a portion of the continuous fabric to be utilized in the manufacturing process may be weighed, in which the results may be extrapolated to a larger quantity of fabric. In such an embodiment, the measured portion may be combined with other parameters of the fabric, such as the width and length of the fabric to determine the final estimate. In an embodiment, ten or more rolls of fabric of fairly similar length can be weighed. The average weight of these rolls of fabric, plus or minus a tolerance for variations, can be used as the general “weight” of each roll.

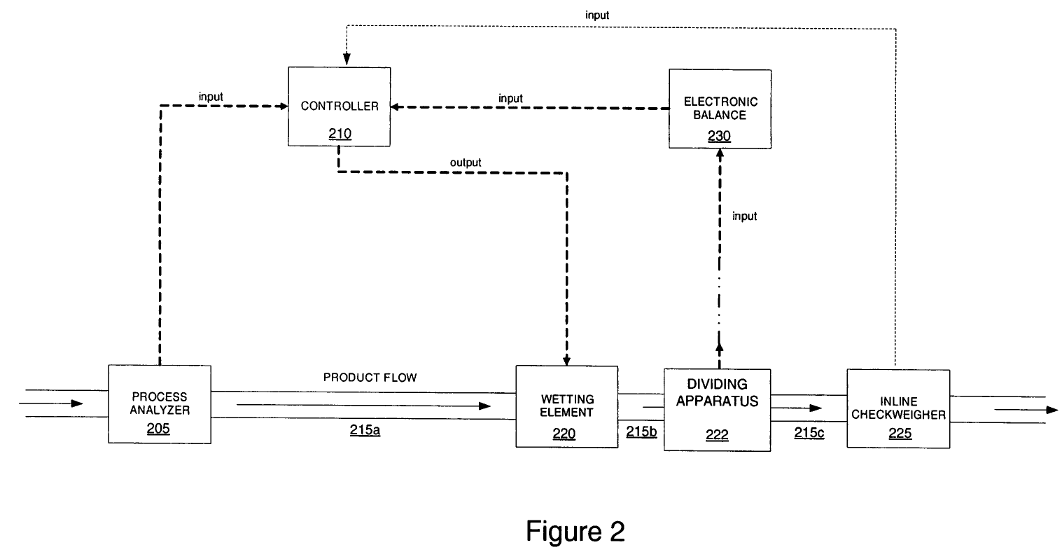

In one embodiment, the weight of a predetermined amount of fabric is determined in step 105. In one embodiment, step 105 may be implemented or aided through a computer-implemented analyzer, such as process analyzer 205. As seen in FIG. 2, process analyzer 205 may be in electronic communication with controller 210. FIG. 2 is a diagram showing an illustrative system for manufacturing a non-woven cloth having a consistent predetermined target dose of solution thereon according to at least one embodiment of the invention. Controller 210 may comprise a computer readable medium for storing the result obtained in step 105, and optionally, storing historical data received from the process analyzer 205 or other components within the manufacturing process. The controller 210 may also comprise a computer readable medium with computer readable instructions for using the results received in 105.

As one skilled in the art will appreciate, there are several methods and procedures that may be implemented to use the results obtained in step 105. In one embodiment, the result may be used to calculate the weight for a specific portion of 'the continuous fabric, such as a desired length of cloth to be produced from the continuous fabric.

In one embodiment, the weight determined in step 105 is used, in whole or in part, to calculate the one or more parameters of the continuous fabric including, but not limited to: the mean weight, the standard deviation, range of weight values, the minimum weight, the maximum weight, and quartiles (such as 1st and 3rd quartile) of the weight. As one skilled in the art will appreciate, a multitude of processes may be used to determine these parameters. In one embodiment, a plurality of computer-implemented instructions, which may be stored on the computer readable medium of a processor or computer are used to obtain one or more of these parameters. The processor or computer may be different from the controller 210 or may be incorporated by the controller 210. In addition, the controller 210 may perform the functions of the processor of computer. Various models may be utilized in combination or individually to determine one or more of these parameters. For example, in one embodiment, as shown in FIG. 4, three or more models may be used collectively to assist in the prediction of the weight. One model or approach, a Confidence Interval (CI) approach, may select a confidence level, such as 95%, to show the upper and lower CI on the mean of the basis weight values as boundary values (e.g., lower and upper limits). This model allows an operator, and or the processor or computer, to predict the mean, or the probable value of the predicted dry cloth weight.

Another method, the Quartile Method, may also be used. Under this method, one or more quartiles, such as 1st and 3rd quartile values, may be utilized as set limits. For example, using the 1st and 3rd quartiles sets the probable value range for at least 50% of the predicted values, or may at least provide a view into the center portion of the distribution. Yet another approach, the Minimum Maximum (Min/Max) approach, may also be utilized to set a maximum and minimum weight values as limits. This model may permit the controller and/or user to estimate the predicted weights at the extreme edges of the distribution.

In yet another embodiment, optional step 110 may be included. During step 110, the width of at least a portion of the predetermined consistent amount of the continuous fabric may be determined. Step 110 may not be performed or may be performed in conjunction with or independent of step 105. In one embodiment employing both step 105 and step 110 the weight and the width of a particular section of the continuous cloth can be estimated before further manufacturing the fabric. In one such embodiment, it may be desirable to produce a cloth from the continuous fabric having a substantially equal length-to-width ratio. In one embodiment, the process analyzer 205 or other electronic or mechanical inputs may transmit an electronic signal to the controller 210 indicating the width or expected width of the continuous fabric. Based upon the received data, the controller may compute the best cutting locations on the substantially continuous fabric to create, for example, a substantially square dimensioned cloth while limiting the least amount of waste (see step 115).

In one embodiment, a tension, such as a back tension, may be applied on the substantially continuous non-woven fabric (step 120). This tension may be based on the result obtained from at least one of the previous steps. In one embodiment, the amount of back tension applied is related to the average width of the continuous fabric. In one such embodiment, an increase in tension results in a decrease in the average width of the fabric, while less tension would result in a larger average width of the fabric. In an embodiment, accurate estimation of the width of the fabric can be dependent on the amount of tension and resulting width of the fabric. In an embodiment, the amount of back tension ranges from approximately 0 to 20% of the line speed or more preferably approximately 8 to 15%.

In an embodiment of the invention, the weight of a non-woven cloth can be predicted with a considerable degree of accuracy. A particular target length and width of a cloth may be desired. In some embodiments, widths and lengths of the cloth ranging from 4 to 24 inches, 6 to 12 inches or 6 to 9 inches may be desired. The cloths may be square, rectangular, circular or any other geometric or non-geometric shape. In an embodiment, the cloth length can be approximately 6⅝-8⅜ inches and the cloth width can be approximately 6.5-8.5 inches. In separate embodiments the cloth lengths and widths can be approximately 7.5 inches by 7.5 inches, approximately 8 inches by 8 inches, approximately 7.5 by 8 inches and approximately 7.5 by 7 inches.

Based on a desired length and width of the cloth, the cloth weight can be predicted with considerable accuracy in some embodiments. In an embodiment, cloth weights can be predicted such that the actual weight of the cloth is within 25% and more preferably within 16% of the desired value in the substantial majority of the cloths.

In an embodiment shown in FIG. 4, basis weights of rolls or lots of fabric are received from a fabric manufacturer. Using the Confidence Interval method, the Quartile method and the Minimum Maximum method, an initial cloth weight is predicted. An initial cloth width is then determined and a desired cloth length is selected. The back tension speed is then adjusted to reach the target desired cloth width. An analysis is then performed to determine whether the clothes maintain the desired level of squareness and at least one cloth is weighed. If the cloth weight is not within an appropriate range of the desired cloth weight, a new cloth length may be selected or the backtension speed may be adjusted until the cloth falls within the appropriate range when weighed. At that point, the weight of the cloth is input into a computer or processor, along with the final settings of the dry weight, cut length and back tension. These parameters in turn can be communicated to the controller 210 where the controller 210 is separate from the computer or processor.

In step 125, a directional movement of the continuous fabric is created, wherein the leading portion of the continuous fabric is followed by the trailing portion of the continuous fabric. As used herein, the term “directional movement” does not limit the fabric to a uniform direction of travel throughout the manufacturing process or even through certain individual steps of the process. Indeed, it is common in the industry for fabrics to move in directions along numerous axes which change throughout the process. The term is merely used to readily convey to the reader that the continuous fabric is moved in a manner that a leading or first portion of the continuous fabric will cross at least one manufacturing point at an earlier time than the trailing or second portion. Moreover, the term is relative to the segment of the continuous fabric being referred to. For example, in one embodiment, as a section of the leading portion is divided in step 140 (described below), and becomes separated from the continuous fabric, the previous trailing portion may become the leading portion or section thereof of the continuous fabric.

In step 130, a liquid solution is applied to the continuous fabric as it is under the directional movement. The solution can be applied in a substantially uniform manner, wherein the solution is first applied to the leading portion of the fabric and subsequently applied to the trailing portion of the continuous fabric, wherein the amount of liquid solution applied to the leading portion of the fabric is substantially equal to the amount of liquid solution applied to the trailing portion. As indicated in FIG. 2, according to one embodiment, the continuous fabric may travel substantially along product flow 215a to a wetting element, such as wetting element 220, at which point the solution is added to the fabric.

In an embodiment the liquid solution is a non-alcohol liquid solution with a base that is primarily water. Other solutions can be used, including solutions with alcohol. Solutions containing alcohol may not exhibit the benefits of non-alcohol solutions. The liquid solutions may, but need not, contain additional chemical compositions, such as propylene glycol, glycerin, aloe vera, dimethicone, igepal CO, polysorbate, shaw mudge fragrance, glucone delta lactone and additional chemical compositions such as stabilizers, surfactants, humectants, skin protectants, buffers and preservatives. In an embodiment, the pH of the solution is approximately 4.2-5.2.

In an embodiment, the liquid solution also may include one or more chemical compositions, such as medications or pharmaceutical preparations. In one such embodiment, the liquid solution comprises chlorhexidine, such as in the form of chlorhexidine gluconate. Other chemical compositions include iodine, provodine iodine, neomycin, alcohol, chloroxylenol and isopropyl alcohol. One skilled in the art will realize that many liquids, regardless of density or viscosity and/or chemical nature may be utilized. Indeed, according to one embodiment, the amount of liquid solution dispersed may be a function of its concentration of active ingredients and/or physical attributes like density and viscosity.

In an embodiment, the solution may contain a coloration, coloring agent or tint. In some applications, health care providers, such as nurses or doctors, desire a visible indicator that a chemical composition has been applied to the skin or other portion of a patient. A coloring agent can be added to the solution such that when the cloth containing the solution is applied to the patient, a portion of the coloration remains on the skin or other portion of the patient after the solution has been applied. The coloring agent may also be added subsequent to or simultaneous with the solution and be added in a predetermined mixed ratio. In addition, in an embodiment, the coloring agent can be added to the substrate prior to the wetting process. The coloring agent may be added during the manufacturing process for the substrate, such as a non-woven fabric. In one embodiment, a chemical composition in the solution may be a disinfectant. After the solution is applied to a patient's skin, the coloration or tint that remains on the skin can be an indicator to a health care provider that the disinfectant has been applied.

The dispersion of fluid may be performed through a plurality of available and known mechanisms. In one embodiment, the wetting element 220 may comprise a substantially rigid surface having at least one opening for placing the liquid solution on the continuous fabric. Yet in another embodiment, the rigid surface comprises a plurality of openings, wherein only a portion of the openings exude the liquid solution based upon the average width of the continuous fabric. In yet another embodiment, a nozzle or other spray device may be used to add a solution to the continuous fabric.

The amount of liquid solution may depend on a myriad of factors. In one embodiment, the dry weight of the fabric, such as that determined in step 105 is used. Another embodiment may utilize the dry weight of the fabric in conjunction with the speed and/or amount of tension placed upon the fabric. As discussed above, a plurality of analytical models may be used to determine the amount of solution to apply. Indeed, in one embodiment, the concentration of at least one chemical composition within the liquid solution is measured or otherwise determined. In one such embodiment, if the concentration of the chemical composition is low, the amount of liquid solution may be increased. Conversely, if the amount of the chemical composition is too high relative to a predetermined limit, the amount of liquid solution applied to the continuous fabric may be decreased.

In step 135, the continuous fabric may be folded along at least one axis after the liquid solution has been applied in step 130. In an embodiment, the fold may be a “z-fold” (sometimes referred to as an “s-fold”), a “c-fold” or other fold style. In one embodiment, the folding of the continuous fabric results in decreasing the width of the fabric to be divided in step 140. Yet in another embodiment, the amount of dispersion of the liquid solution on the fabric is more uniform from increasing the amount of surface area in contact with another portion of the fabric.

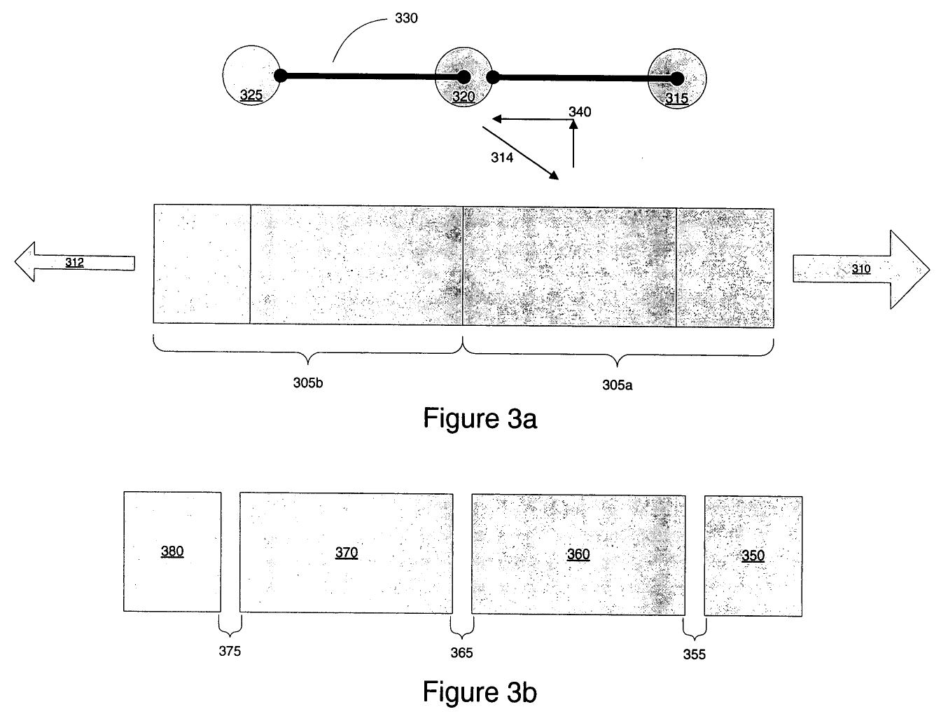

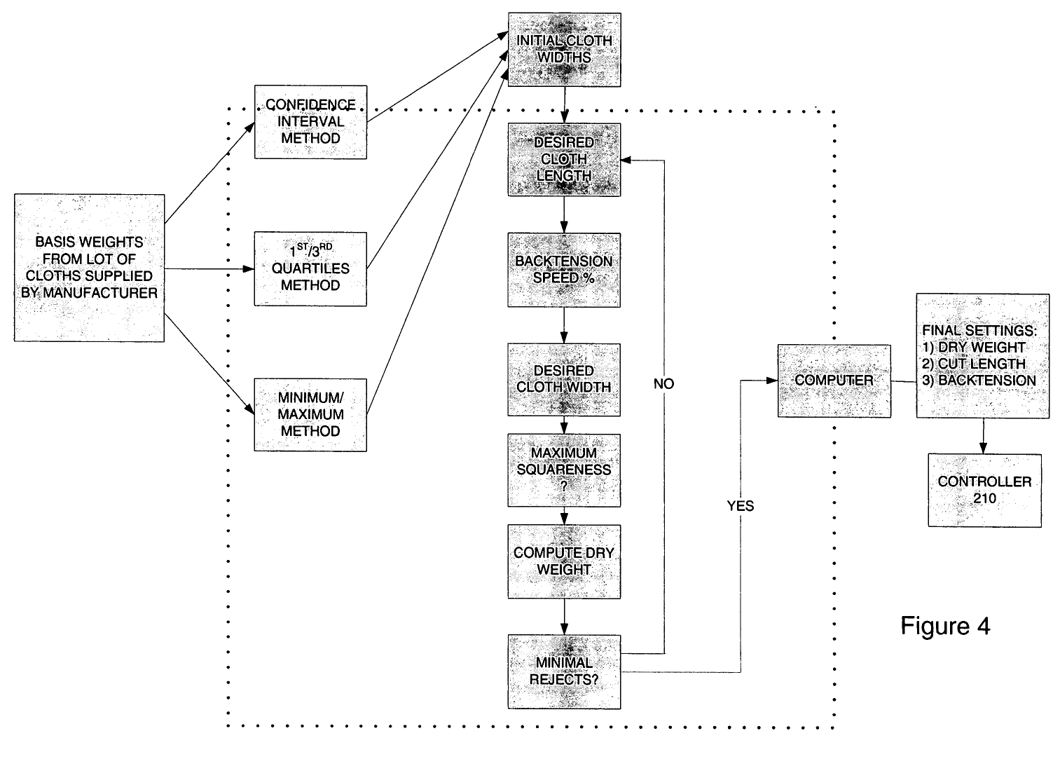

In step 140, while the substantially continuous fabric is under the directional movement, for example, on or about product flow 215b, it is divided into one or more cloths through the substantially simultaneous use of a plurality of cutting devices (see dividing apparatus 222; and FIG. 3a). While the inventors have discovered that a plurality of circular saws disposed on a central spinning axis may achieve the dividing step of the invention, others skilled in the art will realize a wide variety of dividing mechanisms may be utilized, including but not limited to one or more die-cutting plates or rollers, a swinging blade, blades disposed on different mechanisms, the use of large amounts of concentrated or variable pressure, or high powered laser energy.

FIGS. 3a and 3b show the division of the substantially continuous fabric according to at least one embodiment of the invention. As shown in the FIG. 3a, the substantially continuous fabric 305 comprising a leading portion 305a and a trailing portion 305b is traveling under directional movement (indicated by arrow 310) and under a back-tension force (indicated by arrow 312). In the illustrative embodiment, the fabric 305 is traveling under three blades 315, 320, and 325 which may be rigidly attached to and configured to spin on a central axis 330. The speed of the blades on central axis 330 can be adjusted to achieve maximum cutting effectiveness. As one skilled in the art will appreciate, the fabric may be positioned on a different axis in relation to the cutting devices, as long as the cutting devices can divide the fabric.

According to one embodiment of the invention, the dividing apparatus 222 may be configured to travel in a direction substantially similar to the directional movement of the cloth (see arrow 314, showing the blades traveling forward as they cut in a downward fashion). The speed and the movement of the dividing apparatus along the directional movement 310 may be predetermined or adjusted and may be implemented through computer readable instructions stored on one or more computer readable mediums. In one embodiment shown in FIG. 3, the dividing apparatus (indicated by the three blades 315, 320, and 325 on the central axis 330) travel about substantially the same velocity as the fabric 305 along the directional movement 310 as it divides the fabric as indicated by arrow 314. Moreover, while the cutting devices 315, 320, and 325 are shown in a substantially equidistant arrangement, the arrangement may be different in other embodiments.

Upon cutting the fabric 305 (as shown in FIG. 3b), the dividing apparatus may return to its original position. In one embodiment, the dividing apparatus is configured to travel along a different path, such as the path indicated by path 340, as to prevent any further contact with the fabric as it moves along direction 310.

As best observed in FIG. 3b, which shows an illustrative division of the continuous fabric 305 into at least a first cloth and a second cloth through the substantially simultaneous use of a plurality of cutting devices, a leading edge 350 is produced ahead of the division 355 produced by the first cutting device 315. The leading edge may be discarded. In addition, the leading edge may be maintained, especially where the transverse rate of the saw elements is sufficiently high. In an embodiment, the leading edge may be discarded because the width of the leading edge 350 is less uniform due to its position as the leading edge, the presence of back tension on the continuous cloth in the direction of arrow 312 and the transverse rate of the saw elements not being sufficiently high. Substantially simultaneously, a first cloth 360 is produced by divisions 355 and 365 produced by the first cutting device 315 and the second cutting devices 320, respectively.

Similarly, a second cloth is produced by divisions 365 and 375 produced by the second cutting device 320 and the third cutting device 325. As seen in FIG. 3b, a trailing edge 380 is created behind division 375. In at least one embodiment, the trailing edge 380 continues in the direction of travel 310 and becomes the leading portion (such as 305a) of the continuous fabric 305. As one skilled in the art will readily understand, step 140 may be repeated, allowing the fabric to be continually divided into a plurality of cloths.

As one skilled in the art will appreciate, step 140 may be implemented to divide the continuous fabric before step 130 applies the liquid solution to the fabric. In one such embodiment, the individual cloths created in step 140 may be placed substantially adjacent to each other so that the cloths are positioned as to create a uniform surface area as to be substantially similar to a continuous fabric before the fluid is applied in step 130.

In one embodiment of the invention, cloths with a consistent amount of solution can be prepared. The cloths can be made such that the amount of solution in the cloths varies by no more than 20% above or below a pre-established target weight of the solution. In another embodiment, cloths are prepared such that the amount of solution in the cloths varies by no more than 16% above or below a pre-established target weight of the solution. Thus, in one embodiment a cloth can be made such that the pre-determined dose of solution is approximately 25 ml (which will vary up or down from this baseline amount by no more than 16% (i.e., plus or minus 4 ml)).

In an embodiment, cloth with a consistent amount of a chemical composition or other compound can be manufactured. The solution added to the non-woven fabric can contain a chemical composition. These chemical compositions may include chlorohexidine gluconate, beta iodine, among other compositions, alone or in combination. Solutions can be prepared that have a particular concentration of a chemical composition. In an embodiment, cloths with a consistent amount of a chemical composition can be manufactured. Cloths with a consistent amount of chemical composition can be made such that the amount of chemical composition in the cloths varies by no more than 20% above or below a pre-established target weight of the chemical composition. In another embodiment, cloths can be prepared such that the amount of chemical composition added to the cloths varies by no more than 16% above or below a pre-established target weight of the chemical composition solution.

In one embodiment, the amount of chemical composition that is added to a cloth is approximately 500 mg, plus or minus 16% (i.e., plus or minus 80 mg). In other embodiments, chemical composition can be added in amounts ranging from 50 to 1000 mg, including 100, 250, 400 and 750 mg, depending on the desired application. These amounts also can be consistent, ranging by no more than 16% or 20% from a base line (i.e., plus or minus 16% or 20%).

In an embodiment, a chemical composition can be applied to a cloth or fabric in a dry form. The chemical composition can be sprayed on the cloth or fabric as a powder or can settle on the cloth as the cloth passes through a chamber containing airborne particles of the chemical composition. The dry chemical composition can be applied to the cloth such that the amount of chemical composition in the cloths varies by no more than 20%, preferably no more than 16%, above or below a pre-established target weight of the chemical composition.

In an embodiment, the total aerobic count (i.e., the amount of viable air-tolerating microorganisms) of the cloth is less than approximately 500 colony forming units per gram of cloth. The cloth can test negative for e. coli, salmonella, s. aureus and p. aeruginosa in an embodiment.

Step 145 may then be implemented, wherein at least one of the cloths produced in step 140 is weighed. In one embodiment, a predetermined number of cloths may be bound or otherwise positioned for simultaneous weight measurement. For example, as illustrated in FIG. 2, a plurality of individual cloths produced in step 140 may travel substantially along product flow 215c to an inline checkweigher 225. The checkweigher 225 weighs the predetermined number of cloths and in at least one embodiment is in electronic communication with the controller 210. Yet in other embodiments, a single cloth may be weighed, such as through electronic balance 230. In a further embodiment, one or more cloths can be weighed along with a clip, filler, package or other material with known or unknown weight. The weight obtained in step 145 may then be compared with an expected result. In one embodiment, step 150 may be implemented, which, based on the result obtained in step 145, alters the amount of liquid solution dispensed from the wetting element 220 to achieve the predetermined target wet weight. In yet another embodiment, the weight of a portion of continuous fabric may be obtained before step 140 divides the fabric. In one such embodiment, a predetermined amount of fabric may be weighed and compared to a desired value. Indeed, employing such a method may reduce the time and costs associated with dividing the fabric, and thus may allow the manufacturer to dispose of faulty portions before dividing them.

In an embodiment, a substrate, such as a non-woven cloth, containing a solution can be used to reduce skin bacteria. Several days before a surgical procedure (and one day before such a procedure in one embodiment), the substrate containing the solution can be applied to a patient's skin on the site of an upcoming surgical procedure or can be applied to a portion or all of the patient's body. Immediately before the surgical procedure, the substrate can be applied to a portion or all of the patient's body or the portion of the patient's skin at the site of the surgical procedure. In addition, the substrate containing the solution can be applied to the patient's skin or body both immediately before the surgical procedure and hours or days in advance of the procedure. Any invasive procedure, including surgery, introduction of a catheter or any procedure in which the skin is broken are included in the term surgical procedure as used herein

An example of one embodiment of the invention is provided below. This example describes one version of one embodiment of the invention. The invention is not limited to the example described below and includes numerous additional embodiments and versions. The example should not be read to limit the disclosure of the invention in this application.

### EXAMPLE 1

A non-woven cloth can be manufactured from a roll of non-woven fabric that contains 100% polyester fibers. Approximately 60-80% of the polyester fibers have a denier of approximately 1.2 and a length of approximately 1.5 inches. The balance of the fibers have a denier of approximately 4.75 and a length of approximately 3.0 inches. The width of the roll varies somewhat throughout the roll. The roll may be referred to as continuous, even though it is not literally continuous. The length of the fabric in the roll is substantially greater than the width pf the fabric in the roll. Based on previous measurements of non-woven cloths containing the same fibers, the weight of a cloth from the roll with a length of approximately 7.5 inches is estimated to be approximately 5.9-6.0 g.

In one embodiment, a non-alcohol solution containing approximately 2% by weight chlorohexidine gluconate is added to the non-woven fabric in a continuous process. In another embodiment, about 1.8% to about 2.2% by weight chlorohexidine gluconate is added. In yet another embodiment, about 1.0% to about 3.0% by weight chlorohexidine gluconate is added. The process is referred to as continuous, even though the process will stop when the roll of non-woven fabric is completely used and may stop or pause earlier. The fabric from the roll of fabric passes over a wetting agent that contains several nozzles which add the non-alcohol solution to the fabric. The target amount of solution to add to the fabric is 25 grams of solution per 7.5 inches of length of the fabric. The 25 g of solution contains approximately 500 mg of chlorohexidine gluconate. The tolerance for the amount of solution added to the fabric and the amount of chlorohexidine gluconate added to the fabric is 16%. Accordingly, the amount of solution added to each 7.5 inches of the fabric is 25 grams, plus or minus 4 grams. The amount of chlorohexidine gluconate added to each 7.5 inches of the fabric is 500 mg, plus or minus 80 mg.

The non-woven fabric is subjected to a folder that folds the fabric with a “z-fold.” The non-woven fabric is placed on top of another stream of non-woven fabric coming from a separate roll of non-woven fabric. The two layers of non-woven fabric pass through a tension creator which assures that a back tension exists on the fabric. The fabric is then subjected to a series of cuts by three saws. The material downstream from the first saw is discarded. The material (i.e., cloth) between the first saw and the second saw is retained. The material (i.e., cloth) between the second saw and the third saw also is retained. After the cutting, another portion of fabric moves into position to be subjected to the saws and cut into non-woven cloths.

A single cloth after the cutting containing the solution (which contains CHG) is then weighed. If the actual weight is more or less than the target weight, the controller may instruct that more or less solution be added to the corresponding fabric by the wetting agent.

The resulting non-woven cloth contains approximately 25 g (plus or minus 4 g) of solution and contains approximately 500 mg (plus or minus 80 mg) of CHG. In a substantially continuous process, substantially all of the cloths produced will contain approximately 25 g (plus or minus 4 g) of solution and contain approximately 500 mg (plus or minus 80 mg) of CHG.

Variations and modifications of the foregoing are within the scope of the present invention. It should be understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention. The claims are to be construed to include alternative embodiments to the extent permitted by the prior art.

1. A method for manufacturing a non-woven cloth containing a consistent dose of solution thereon comprising the steps of:
(a) determining the weight of a predetermined amount of continuous non-woven fabric, the fabric having a leading portion and a trailing portion;
(b) creating directional movement of the continuous fabric, wherein the leading portion of the continuous fabric is followed by the trailing portion of the continuous fabric;
(c) applying a liquid solution to the continuous fabric as it is under the directional movement, the liquid solution first being applied to the leading portion of the fabric and sequentially applied to the trailing portion of the continuous fabric, wherein the amount of liquid solution applied to the leading portion of the fabric is substantially equal to the amount of liquid solution applied to the trailing portion;
(d) while the continuous fabric is under the directional movement, dividing the continuous fabric into at least a first cloth and a second cloth through the substantially simultaneous use of a plurality of cutting devices, wherein along the direction of movement, a leading edge is produced ahead of the first cutting device, a first cloth is produced between the first cutting device and the second cutting devices, the second cloth being produced between the second cutting device and the third cutting device, and a trailing edge is produced following a last cutting device; and
(e) weighing at least one of the cloths produced in step (d).

2. The method of claim 1, further comprising the step of:
(f) based upon the weight received in step (e), adjusting the amount of liquid solution applied to the continuous fabric in step (c).

3. The method of claim 1, further comprising the step of:
(g) based upon the weight determined in step (a), adjusting the amount of liquid solution applied to the continuous fabric in step (c).

4. The method of claim 1, further comprising the step of:
(h) folding the continuous fabric along at least one axis after the liquid solution has been applied in step (c).

5. The method of claim 1, further comprising the step of:
(i) prior to cutting the continuous fabric in step (c), applying pressure to the continuous fabric to create tension of the fabric, wherein the tension creates a substantially determinable extension of the continuous fabric in at least one dimension.

6. The method of claim 5, further comprising the steps of:
(j) determining the concentration of at least one chemical composition within the liquid solution; and
(k) adjusting the amount of liquid solution applied to the continuous fabric in step (c).

7. The method of claim 2, wherein the first and second cloths are substantially the same weight.

8. The method of claim 1, wherein the step (c) comprises passing the continuous fabric against a substantially rigid surface having at least opening, wherein the liquid solution is placed from at least one opening onto the continuous fabric as the fabric passes against the rigid surface.

9. The method of claim 1, wherein the liquid solution comprises chlorohexidine gluconate.

10. An article of manufacture produced by the method of claim 1.

11. A method of manufacturing non-woven cloths containing a consistent amount of solution, comprising:
(a) determining the approximate weight of a non-woven cloth before the solution in step (c) has been added,
(b) applying directional movement to a substantially continuous amount of non-woven fabric,
(c) adding a solution to the substantially continuous amount of non-woven fabric in a continuous process,
(d) cutting the substantially continuous amount of non-woven fabric into a plurality of non-woven cloths, wherein each of the non-woven cloths has a similar shape and size,
(e) weighing at least one of the plurality of non-woven cloths after the solution in step (c) has been added,
(f) determining whether to adjust the amount of solution that is added to the continuous amount of non-woven fabric based at least in part on whether the weight of the at least one of the plurality of non-woven cloths after the solution in step (c) has been added falls outside a pre-determined range of acceptable weights,
wherein substantially all of the plurality of non-woven cloths contain an amount of the solution that varies by no more than 20% above or below a pre-established target weight of the solution.

12. The method of claim 11 further comprising the step of:
(g) prior to cutting the continuous fabric in step (d), creating tension in the substantially continuous amount of fabric, wherein the tension decreases the width of the substantially continuous amount of fabric in relation to its length.

13. The method of claim 11 wherein substantially all of the plurality of non-woven cloths contain an amount of the solution that varies by no more than 16% above or below the pre-established target weight of the solution.

14. The method of claim 13 wherein the solution is a non-alcohol solution.

15. The method of claim 11 wherein the non-woven cloths comprise polyester fibers and wherein approximately 60-80% of the polyester fibers have a denier of approximately 1.0-1.4 and a length of approximately 1.2-1.8 inches and wherein approximately 20-40% of the polyester fibers have a denier of approximately 4.5-5.0 and a length of approximately 2.7-3.3 inches.

16. An article of manufacture produced by the method of claim 15.

17. A method of manufacturing substrates containing a consistent amount of solution, comprising:
(a) determining the approximate weight of a substrate before the solution in step (c) has been added,
(b) applying directional movement to a substantially continuous amount of substrate,
(c) adding a solution to the substantially continuous amount of substrate in a continuous process,
(d) cutting the substantially continuous amount of substrate into a plurality of individual subtsrates, wherein each of the individual substrates has a similar shape and size,
(e) weighing at least one of the plurality of individual substrates after the solution in step (c) has been added,
(f) determining whether to adjust the amount of solution that is added to the continuous amount of substrate based at least in part on whether the weight of the at least one of the plurality of individual substrates after the solution in step (c) has been added falls outside a pre-determined range of acceptable weights,
wherein substantially all of the plurality of individual substrates contain an amount of the solution that varies by no more than 20% above or below a pre-established target weight of the solution.

18. A method of manufacturing non-woven cloths, comprising:
(a) determining the weight of a non-woven cloth of substantially a particular dimension,
(b) applying directional movement to a non-woven fabric,
(c) applying a solution to the non-woven fabric,
(d) cutting the non-woven fabric into a plurality of non-woven cloths of substantially the particular dimension,
wherein the solution comprises water and a chemical composition and wherein substantially all of the plurality of non-woven cloths contain an amount of the chemical composition that varies by no more than 20% over or below a predetermined target value.

19. The method of claim 18 wherein the chemical composition is chlorohexidine gluconate and wherein substantially all of the plurality of cloths contain an amount of chlorohexidine gluconate that varies by no more than 16% over or below the predetermined target value.

20. The method of claim 18 wherein substantially all of the plurality of cloths include approximately 21 to 29 g of chlorohexidine gluconate.

21. A method for manufacturing a non-woven cloth having a consistent pre-determined dose of solution thereon comprising the steps of:
(a) determining the weight of a predetermined amount of non-woven fabric, the fabric having a leading portion and a trailing portion;
(b) determining the width of at least a portion of predetermined amount of non-woven fabric;
(c) comparing the determined weight obtained in step (a) with a predetermined standard weight;
(d) based on the result obtained in step (c ); applying a back tension on the non-woven fabric, wherein the amount of back tension applied is related to the average width of the fabric;
(e) creating directional movement of the fabric, wherein the leading portion of the cfabric is followed by the trailing portion of the fabric under substantially the amount of back tension applied in step (d);
(f) applying a liquid solution to the fabric as it is under the directional movement, the liquid solution first being applied to the leading portion of the fabric and sequentially applied to the trailing portion of the fabric, wherein the amount of liquid solution applied to the leading portion of the fabric is substantially equal to the amount of liquid solution applied to the trailing portion;
(g) while the fabric is under the directional movement, dividing the fabric into a at least a first cloth and a second cloth through the substantially simultaneous use of a plurality of cutting devices, wherein along the direction of movement, a leading edge is produced ahead of the first cutting device, a first cloth is produced between the first cutting device and the second cutting devices, the second cloth being produced between the second cutting device and the third cutting device, and a trailing edge is produced following a last cutting device; and
(h) weighing at least one of the cloths produced in step (g).

22. The method of claim 21, wherein the average width of the cloths and the average length of the cloths is about equal.

23. The method of claim 21, wherein the step (f) comprises passing the fabric against a substantially rigid surface having at least opening, wherein the liquid solution is expelled from at least one opening onto the fabric as the fabric passes against the rigid surface.

24. The method of claim 23, wherein the rigid surface comprises a plurality of openings, wherein only a portion of the openings expel the liquid solution based upon the result obtained in step (c).

25. The method of claim 21, wherein the continuous fabric comprises polyester fibers and wherein approximately 60-80% of the polyester fibers have a denier of approximately 1.2 and a length of approximately 1.5 inches and wherein approximately 20-40% of the polyester fibers have a denier of approximately 4.75 and a length of approximately 3.0 inches.

26. The method of claim 21, wherein the liquid solution comprises chlorohexidine gluconate.

27. An article of manufacture produced by the method of claim 21.
